(19) United States
(12) Patent Application Publication
(10) Pub. No.: US 2006/0228785 A1
(43) Pub. Date: Oct. 12, 2006

(54) Recombinant microorganism for the production of vitamin b6

(76) Inventors: Tatsuo Hoshino, Kanagawa (JP); Keiko Ichikawa, Kanagawa (JP); Masaaki Tazoe, Kanagawa (JP)

Correspondence Address: Stephen M Haracz;Bryan Cave, 1290 Avenue of the Americas, New York, NY, 10104-3300, US

(21) Appl. No.: 10/528,891

(22) PCT Filed: Sep. 18, 2003
(86) PCT No.: PCT/EP03/10403

§ 371 Date: Mar. 31, 2006

(30) Foreign Application Priority Data
Sep. 27, 2002 (EP) 02021623.0

(51) Int. Cl. C12P 19/42; C12N 1/21

(52) U.S. Cl. 435/86; 435/252.33

(57) ABSTRACT

Disclosed is a recombinant microorganism being capable of producing vitamin B6, wherein said microorganism carries extra genes which code for an enzyme combination selected from: i) erythrose 4-phosphate dehydrogenase and 1-deoxy-D-xylulose 5-phosphate synthase; ii) erythrose 4-phosphate dehydrogenase and pyridoxol 5′-phosphate synthase; and iii) erythrose 4-phosphate dehydrogenase, 1-deoxy-D-xylulose 5-phosphate synthase and pyridoxol 5′-phosphate synthase.

The present invention relates to a recombinant microorganism and a process for preparing vitamin B₆ by using the same.

“Vitamin B₆” as used in the present invention includes pyridoxol, pyridoxal, and pyridoxamine. Vitamin B₆ is a vitamin indispensable to human beings or other animals and used as a raw material of medicines or as feed additives.

The present invention provides recombinant microorganisms carrying the cloned genes for over-expression of the enzymes involved in the vitamin B₆ biosynthetic pathway to produce vitamin B₆.

Examples for suitable microorganisms include members of the genus Escherichia, capable of over-producing vitamin B₆, e.g. E. coli AT1024 or WG497.

The present invention also provides a method to prepare vitamin B₆ effectively by cultivating said recombinant microorganism resulting in accumulation of multipliable amounts of vitamin B₆ in culture broth and to separate vitamin B₆ from the culture broth.

The present invention also provides a method for preparing vitamin B₆ by cultivating a recombinant microorganism, which is expressing the cloned genes encoding enzymes involved in the vitamin B₆ biosynthetic pathway, in appropriate medium, and by separating vitamin B₆ from the culture broth.

Preferably the recombinant microorganism carries an extra nucleic acid encoding an enzyme or an enzyme combination selected from

i) erythrose 4-phosphate dehydrogenase (E4P dehydrogenase),

ii) E4P dehydrogenase and 1-deoxy-D-xylulose 5-phosphate synthase (DXP synthase),

iii) E4P dehydrogenase and pyridoxol 5′-phosphate synthase (PNP synthase), and

iv) E4P dehydrogenase, DXP synthase and PNP synthase.

In the vitamin B₆ biosynthesis in E. coli PNP (pyridoxol 5′-phophate) is synthesized de novo by the condensation of two intermediates, 4PHT (4-phosphohydroxy-L-threonine) and DXP (1-deoxy-D-xylulose-5-phophate), catalyzed by 4PHT dehydrogenase and PNP synthase. 4PHT is synthesized via a pathway leading from E4P (erythrose 4-phosphate) by a three-step reaction: dehydrogenation of E4P by E4P dehydrogenase; dehydrogenation of 4-phospho-erythronate catalyzed by 4-phospho-erythronate dehydrogenase; and amination of 3-hydroxy-4-phosphohydroxy-α-ketobutyrate catalyzed by phosphoserine amino-transferase. DXP is synthesized by a condensation of glyceraldehyde 3-phosphate and pyruvate with DXP synthase.

In these pathways, any step can be a bottleneck. In developing an industrial process for producing vitamin B₆, the amount of the vitamin produced can be increased, e.g., by overcoming the bottlenecks. This can be achieved by, e.g., increasing the amount of enzyme on the reaction. The present invention also relates to the cloning and over-expressing of genes coding for enzymes involved in the vitamin B₆ pathway in a host cell. Increasing the amount of an enzyme can be achieved, e.g., by introduction of desired genes in expressible form, e.g., using vectors replicable in a host cell, and additionally (i) by increasing the intended gene copy number, e.g., using a plasmid with an increased copy number, and/or (ii) by increasing transcription, e.g., by using promoter elements, and/or (iii) by increasing the rate of translation, e.g., by using a consensus ribosome binding site. Combinations of additional (i), (ii), and/or (iii) can also be employed for increasing the amount of an enzyme involved in the vitamin B₆ biosynthetic pathway.

In a preferred embodiment of the invention, more than one gene of the vitamin B₆ biosynthetic pathway was introduced into the microorganisms.

The appropriate genes may be isolated in accordance with known methods. The complete nucleotide sequences of E. coli K-12 (ATCC 25404) are known [Blattner et al., Science 277:1453-1474 (1997)] and deposited in databases. When the E. coli genes relevant to vitamin B₆ biosynthesis are being cloned, the polymerase chain-reaction (PCR) method is suitable.

For PCR, at the 5′ end of the gene, primers are designed to amplify the entire protein-coding-region including the initiation codon and the ribosome binding site preceding it. At the 3′ ends of the genes, the codons responsible for termination of protein synthesis are always included in the amplified segment, but any transcription termination signals are excluded. All primers are modified by addition of the recognition sequences for specific restriction endonucleases at the 5′ end of each primer.

According to the present invention, enhanced expression of genes coding for enzymes responding for biosynthesis of vitamin B₆ is mediated by transferring the corresponding genes into a host cell in expressible form. The genes may be chromosomal (e.g. integrated into a host cell chromosome by homologous recombination or other mechanism) or extrachromosomal (e.g. carried by plasmids, cosmids, phages and the like).

The genes can be introduced into a host cell by plasmids, cosmids, phages, or other vectors that mediate transfer of genes into a host cell. Selectable markers can be present on the vector to aid in identification of host cells into which the genes have been introduced. Examples for selectable markers are genes that confer resistance to particular antibiotics, such as tetracycline, ampicillin, chloramphenicol, kanamycin, or neomycin (referred to as Tc, Ap, Cm, Km, and Nm, respectively, hereinafter).

A means for introducing genes into a host cell may use an extrachromosomal multi-copy plasmid vector into which genes have been inserted. Plasmid-borne introduction of the genes into host cells involves an initial cleaving of a plasmid with a restriction enzyme, followed by ligation of the plasmid and genes. Upon recircularization of the ligated recombinant plasmid, transfer into the host cell may be carried out by methods well known in the art such as electroporation, calcium-dependent transformation, and conjugation. Plasmids suitable for insertion of genes into the host cell include, but are not limited to, pBR322 and its derivatives such as pKK223-3, pUC vectors, and pACYC and its derivatives such as pSTV29. In addition, cosmid vectors such as pVK100 are also suitable for the insertion of the genes into host cells.

The amplified gene can be placed on a vector capable of being expressed in a host cell, and can be transformed into a microorganism producing vitamin B₆ in accordance with current methods. In the case of introducing the other gene into a microorganism as the second gene in this invention, the gene can be placed on the other vector, which is compatible to the present plasmid in the host cell, and can be transformed into a microorganism which already carries the first plasmid.

Thus-obtained microorganisms may be cultivated in a medium containing assimilable carbon sources, digestible nitrogen sources, inorganic salts and other nutrients necessary for growth of the microorganism. As the carbon source, e.g., glucose, fructose, lactose, galactose, sucrose, maltose, starch, dextrin or glycerol may be employed. As the nitrogen source, e.g., peptone, soybean powder, corn steep liquor, yeast extract, meat extract, ammonium sulfate, ammonium nitrate or mixtures thereof may be employed. Inorganic salts, sulfates, hydrochlorides or phosphates of calcium, magnesium, zinc, manganese, cobalt and iron may be employed. Conventional nutrient factors or an antifoaming agent such as animal oil, vegetable oil or mineral oil can also be present. The pH of the culture medium is suitably in a range of from about 5.0 to about 9.0, preferably 6.5 to 7.5. The cultivation temperature is suitably in a range of from about 10° C. to 40° C., preferably 34° C. to 37° C. The cultivation time is suitably about 1 day to 7 days, preferably 2 days to 3 days. In the cultivation, aeration and agitation usually give favorable results.

The amount of vitamin B₆ produced in culture broth can be assayed by the turbidity method with Saccharomyces carlsbergensis ATCC 9080. Vitamin B₆ derivatives such as pyridoxol, pyridoxal, and pyridoxamine can be separately quantified by high pressure liquid chromatography (referred to as HPLC hereinafter).

Vitamin B₆ can be collected from the culture broth, e.g., by separating and removing the cells, subjecting to ion exchange resin treatment, concentration cooling crystallization, membrane separation, and other known methods in any suitable combination. In order to remove impurities, activated carbon adsorption and re-crystallization may be used for purification.

The invention is explained in more detail below with the aid of a few implementation examples.

General Methods

E. coli AT1024 is freely available from CGSC under number 4559.

In the genetic studies, strains of E. coli were, unless otherwise indicated, cultured on LB medium consisting of 1% Bacto Tryptone (Becton Dickinson Microbiology systems, MD, USA), 0.5% Bacto Yeast extract (Becton Dickinson Microbiology systems, MD, USA) and 0.5% NaCl. Depending on the resistance properties of the strains employed, Ap (100 μg/ml), Cm (100 μg/ml), Tc (10 μg/ml) or a mixture thereof was added to the medium if necessary. For this, Ap was dissolved in water, Tc in 50% ethanol, Cm in ethanol, and the solutions were added, after having been sterilized by filtration, to the previously autoclaved medium. Bacto-agar (1.5%) was added to the LB medium for preparing agar plates. Plasmid DNA was isolated from E. coli with QIAGEN Midi kit (QIAGEN GmbH, Germany) or with Automatic DNA Isolation System PI-50 (Kurabo Industry Ltd., Japan). Chromosomal DNA was isolated from E. coli K-12 using QIAGEN genomic-tips (QIAGEN GmbH, Germany).

Restriction enzymes, alkaline phosphatase, ligation kit (Takara Bio; Inc, Shiga, Japan), TOPO TA cloning kit (Invitrogen Japan K.K., Japan), and KOD Dash (Toyobo Co., Ltd., Japan) were used in accordance with the producers' instructions. For restriction enzyme analysis, the DNA fragments were fractionated in agarose gels (1.0%) and isolated from the gels by means of extraction using a commercially available system with QIAEXII (QIAGEN GmbH, Germany). DNA sequence was determined with an ALF DNA sequencer (Amersham Biosciences Corp., NJ, USA).

For transformation, the cells were incubated with a shaking of 160 rpm at 37° C. for 2.5-3 h in LB medium (50 ml in 200-ml flasks). At an optical density (600 nm) of approx. 0.4, the cells were spun down and taken up in one tenth the volume of 0.1M MgCl₂. After an incubation of 30 min at 4° C. with from 0.1 to 100 ng of DNA, and subsequent incubation at 37° C. for 1 hour, the cells were plated out on LB medium containing appropriate antibiotics.

### EXAMPLE 1

### Construction of Recombinant Plasmid pKK-epd

The epd gene was amplified from 100 ng of chromosomal DNA of E. coli K-12 with advantage-HF PCR kit using 10-pmol of two primers, SEQ ID NO:1 and 2. Reaction conditions were as follows; after holding 15 sec at 94° C., 25 cycles of 15 sec at 94° C., 3 min at 68° C. The amplified 1.0-kb fragment was directly cloned in pCRII-TOPO vector with TOPO TA cloning kit. Sequence of amplified region was ascertained to be identical with the CDS region of epd (3070692-3071711, complement) in accession number NC_{—}000913. The DNA corresponding to amplified region was cut out by digestion with PstI and recovered from agarose gel. This DNA was ligated to pKK223-3 expression vector (Amersham Biosciences Corp., NJ, USA) that has been opened with PstI and dephospharylated. After transformation of E. coli JM109 competent cells (Takara Bio. Inc, Shiga, Japan), plasmids of transformants were prepared and analyzed with restriction enzyme. A recombinant plasmid pKK-epd, wherein epd gene was inserted into the PstI site of pKK223-3 as the same direction of tac promoter on the vector, was obtained. Plasmid pKK-epd was prepared with E. coli JM109/pKK-epd with QIAGEN Midi kit.

### EXAMPLE 2

### Construction of Recombinant Plasmid pKK-serC

The serC gene of E. coli K-12 was amplified as described in Example 1, except using two primers, SEQ ID NO:3 and 4. The resulting 1.1-kb PCR product was inserted into pCRII-TOPO. It was ascertained that the sequence of the amplified region was identical with the CDS region of serC (956876-957964) in accession number NC_{—}000913. The DNA corresponding to the amplified region was inserted into pKK223-3 yielding pKK-serC as in Example 1 except for digestion with SmaI. Plasmid pKK-serC was prepared with E. coli JM109/pKK-serC with QIAGEN Midi kit.

### EXAMPLE 3

### Construction of Recombinant Plasmid pKK-dxs

The dxs gene of E. coli K-12 was amplified as in Example 1, except using two primers, SEQ ID NO:5 and 6. The resulting 1.9-kb PCR product was inserted into pCRII-TOPO, and the sequence of the amplified region was ascertained to be identical with the CDS region of dxs (437539-439401, complement) in accession number NC_{—}000913. The DNA corresponding to amplified region was inserted into pKK223-3 yielding pKK-dxs as in Example 1 except for digestion with EcoRI. Plasmid pKK-dxs was prepared with E. coli JM109/pKK-dxs with QIAGEN Midi kit.

### EXAMPLE 4

### Construction of Recombinant Plasmid pKK-pdxB

The pdxB gene of E. coli K-12 was amplified as in Example 1 except of using two primers, SEQ ID NO:7 and 8. Reaction conditions were as follows; after holding 15 sec at 94° C., 25 cycles of 15 sec at 94° C., 1 min at 58° C., 1 min at 72° C. The resulting 1.15-kb PCR product was inserted into pCRII-TOPO, and sequence of amplified region was ascertained to be identical with the CDS region of pdxB (2434735-2435871, complement) in accession number NC_{—}000913. The DNA corresponding to amplified region was inserted into pKK223-3 yielding pKK-pdxB as in Example 1 except for digestion with EcoRI. Plasmid pKK-pdxB was prepared from E. coli JM109/pKK-pdxB with QIAGEN Midi kit.

### EXAMPLE 5

### Construction of Recombinant Plasmid pKK-pdxJ

The pdxJ gene of E. coli K-12 was amplified as the same procedure as described in Example 4, except using two primers, SEQ ID NO: 9 and 10. The resulting 0.75-kb PCR product was inserted into pCRII-TOPO, and that sequence of amplified region was ascertained to be identical with the CDS region of pdxJ (2699018-2699749, complement) in accession number NC_{—}000913. The DNA corresponding to amplified region was inserted into pKK223-3 yielding pKK-pdxJ as in Example 1 except for digestion with HindIII. Plasmid pKK-pdxJ was prepared with E. coli JM109/pKK-pdxJ with QIAGEN Midi kit.

### EXAMPLE 6

### Construction of Recombinant Plasmid pKK-pdxA

The pdxA gene of E. coli K-12 was amplified by PCR from 100 ng of chromosomal DNA of E. coli K-12 with KOD Dash using 10-pmol of two primers, SEQ ID NO: 11 and 12. Reaction conditions were as follows; after holding 1 min at 94° C., 30 cycles of 30 sec at 94° C., 2 sec at 48° C., 30 sec at 74° C. The resulting 1.0-kb PCR product was inserted into pCRII-TOPO, and that sequence of amplified region was ascertained to be identical with the CDS region of pdxA (52427-53416, complement) in accession number NC_{—}000913. The DNA corresponding to amplified region was cut out by double digestion with MunI and EcoRI and recovered from agarose gel. The DNA fragment was inserted into pKK223-3 yielding pKK-pdxA as in Example 1 except for digestion with EcoRI. Plasmid pKK-pdxA was prepared with E. coli JM109/pKK-pdxA with QIAGEN Midi kit.

### EXAMPLE 7

### Construction of Recombinant Plasmid pVK-pdxJ

Plasmid pKK-pdxJ obtained in Example 5 was digested with ScaI and SphI. Resulting 2-kb fragment containing tac promoter and pdxJ was purified from agarose gel. Plasmid pVK100 was prepared from E. coli HB101/pVK100, digested with BglII, blunt-ended with blunting kit and dephosphorylated. The 2-kb fragment was ligated to thus-obtained pVK100. E. coli HB101 competent cells (Takara Bio Inc., Shiga, Japan) were transformed with this ligation mixture and plasmids of transformants were analyzed with restriction enzyme. A recombinant plasmid pVK-pdxJ, wherein tac promoter and pdxJ gene were inserted into the BglII site of pVK100 as the same direction of Km resistant gene, was obtained. Plasmid pVK-pdxJ was prepared with E. coli HB101/pVK-pdxJ with QIAGEN Midi kit.

### EXAMPLE 8

### Construction of Recombinant Plasmid pVK-dxs

Plasmid pKK-dxs obtained in Example 3 was digested with BamHI and resulting 2.2-kb fragment containing tac promoter and dxs was purified from agarose gel. Plasmid pVK100 was cleaved with BglII and dephosphorylated. The 2.2-kb fragment was ligated to thus-obtained pVK100 yielding pVK-dxs, wherein tac promoter and dxs gene were inserted into the BglII site of pVK100 as the same direction of Km resistant gene, as in Example 7. Plasmid pVK-dxs was prepared with E. coli HB101/pVK-dxs with QIAGEN Midi kit.

### EXAMPLE 9

### Construction of Recombinant Plasmid pSTV-dxs

The 2.2-kb fragment obtained in Example 8 was ligated to pSTV29 (TaKaRa Bio Inc., Japan) that had been cleaved with BamHI and dephosphorylated. A recombinant plasmid pSTV-dxs, wherein tac promoter and dxs gene were inserted into the BamHI site of pSTV29 as the same direction of lacZ gene, was obtained as in Example 7. Plasmid pSTV-dxs was prepared with E. coli HB101/pSTV-dxs with QIAGEN Midi kit.

### EXAMPLE 10

### Preparation of Microorganisms Harboring Recombinant Plasmids

E. coli AT1024/pKK-epd, E. coli AT1024/pKK-serC, E. coli AT1024/pKK-dxs, E. coli AT1024/pKK-pdxB, E. coli AT1024/pKK-pdxJ and E. coli AT1024/pKK-pdxA were prepared by transformation of plasmids pKK-epd, pKK-serC, pKK-dxs, pKK-pdxB, pKK-pdxJ and pKK-pdxA, respectively, into E. coli AT1024. E. coli AT1024/pKK-pdxJ and pVK-dxs was prepared by transformation of plasmid pVK-dxs into E. coli AT1024/pKK-pdxJ. E. coli AT1024/pKK-epd and pVK-pdxJ was prepared by transformation of plasmid pVK-pdxJ into E. coli AT1024/pKK-epd. E. coli AT1024/pKK-epd and pVK-dxs was prepared by transformation of plasmid pVK-dxs into E. coli AT1024/pKK-epd. E. coli AT1024/pKK-epd, pVK-pdxJ and pSTV-dxs was prepared by transformation of plasmid pSTV-dxs into E. coli AT1024/pKK-epd and pVK-pdxJ. Each recombinant strain was stored as frozen stock made as follows. Each recombinant strain was cultured overnight in liquid LB medium with appropriate antibiotics. Cells were harvested, washed with saline, suspended in sterile 15% glycerol solution at OD₆₀₀=5 and stored in a deep freezer at −120° C. When needed, the frozen stock was thawed before use.

### EXAMPLE 11

### Production of Vitamin B₆ by Recombinant E. coli

Recombinant E. coli strains were cultured as follows. Each 45 μl of these frozen stock prepared in Example 10 was inoculated into a tube containing 5 ml of seed medium [10 g/L of glycerol, 10 g/L of Bacto Tryptone, 5 g/L of Bacto Yeast extract, 5 g/L of NaCl (pH not adjusted)] containing appropriate antibiotics. After shaking tubes for 16 hours at 37° C., each 0.1 ml of the culture was transferred into a flask containing 50 ml of PY30 medium (20 g/L of glycerol, 10 g/L of Bacto Tryptone, 5 g/L of Bacto Yeast extract, 5 g/L of NaCl, 200 mg/L of MgSO₄.7H₂O, 10 mg/L of FeSO₄.7H₂O, 10 mg/L of MnSO₄.5H₂O, pH6.8) with appropriate antibiotics. Flasks were shaken at 37° C. at 180 rpm. After cultivation for 31 hours, amount of vitamin B₆ in supernatant of the culture broth was assayed by the turbidity method with S. carlsbergensis ATCC 9080 as described below. The supernatants of culture broth and standard solutions of pyridoxol hydrochloride (0-100 mg per liter) were serially diluted to 2.09×10⁻⁴ in distilled water. 100 μl of the diluted solution, 1.5 ml of distilled water and 40 μl of 1N H₂SO₄ were added to tubes in this order. After autoclaving at 120° C. for 20 min, 1.5 ml of sterilized assay medium for vitamin B₆ (Nissui Seiyaku Co., Japan) containing S. carlsbergensis ATCC 9080 at OD₆₀₀=0.028 was added to the tubes. The tubes were placed with an incline of 30° and incubated without shaking at 28° C. for 17 hours. The growth of cells was stopped by adding 5 ml of 0.2 N hydrochloric acid, and then the absorbance of the samples was measured at 660 nm with UV-2200 spectrophotometer (shimadzu Co. Ltd., Japan). The amount of vitamin B₆ in a sample was determined by comparing the turbidity of the sample with standard growth curve of S. carlsbergensis ATCC 9080.

Concentrations of vitamin B₆ produced with recombinant E. coli strains are shown in Table 1. Among recombinant E. coli strains carrying single extra plasmid, only three strains showed increased level of vitamin B₆. Namely, the recombinant strains harboring the pKK-epd plasmid, the pKK-pdxJ plasmid, and the pKK-dxs plasmid accumulated vitamin B₆ at 14.2 mg/L, 5.1 mg/L, and 3.9 mg/L, respectively; these are 7.1-fold, 2.55-fold, and 1.95-fold higher than vitamin B₆ accumulation of the host strain, E. coli AT1024. Further, amount of accumulated vitamin B₆ could be raised to 49.2 mg/L or 57.9 mg/L by introduction of two plasmids according to the invention, pKK-epd and pVK-dxs, or pKK-epd and pVK-pdxJ, whereas the amount of accumulated vitamin B₆ was 5.4 mg/L by introduction of pKK-pdxJ and pVK-dxs. In other words, among recombinant E. coli strains carrying a combination of two extra plasmids, only the combination which contains epd showed marked increase in the amount of vitamin B₆, suggesting the synergistic effects of the combination containing epd. Moreover, by introduction of three plasmids, pKK-epd, pVK-pdxJ and pSTV-dxs into one host cell, amount of vitamin B₆ was raised further to 78.5 mg/L. This corresponds to an increase of 39.3-fold compared with host strain.

These results show synergistic effects of introducing epd, pdxJ, and dxs into one host cell on vitamin B₆ production in E. coli AT1024.

TABLE I

| | vitamin B₆ | factor of |
|---|---|---|
| microorganism | (mg/L) | increase |
| E. coli AT1024 | 2.0 | 1.0 |
| E. coli AT1024/pKK-serC | 1.3 | 0.65 |
| E. coli AT1024/pKK-pdxA | 1.5 | 0.75 |
| E. coli AT1024/pKK-pdxB | 2.0 | 1.0 |
| E. coli AT1024/pKK-dxs | 3.9 | 1.95 |
| E. coli AT1024/pKK-pdxJ | 5.1 | 2.55 |
| E. coli AT1024/pKK-epd | 14.2 | 7.1 |
| E. coli AT1024/pKK-pdxJ/pVK-dxs | 5.4 | 2.7 |
| E. coli AT1024/pKK-epd/pVK-pdxJ | 57.9 | 28.9 |
| E. coli AT1024/pKK-epd/pVK-dxs | 49.2 | 24.6 |
| E. coli AT1024/pKK-epd/pVK-pdxJ/pSTV-dxs | 78.5 | 39.3 |

### EXAMPLE 12

### Separation of Vitamin B₆ from Cultural Broth

Produced vitamin B₆ was recovered from the culture broth of E. coli AT1024/pKK-epd, pVK-pdxJ and pSTV-dxs prepared in the same cultural conditions as described in Example 11. Pyridoxol at each purification step and the concentration was followed by HPLC as described below. 50 pd of the solution containing 100 mg/l of 4′-deoxypyridoxol hydrochloride as internal substance was added to 200 μl of the standard solutions of pyridoxol hydrochloride or the sample, and then the mixture was analyzed as follows. The analytical conditions were: column: Capcell pak C18 SG120 (4.6×250 mm) (Shiseido Co., Ltd., Tokyo, Japan); mobile phase: 0.1 M sodium perchlorate, 0.1 M potassium phosphate, and 2% acetonitrile (pH 3.5); column temperature: 25-26° C.; flow rate: 1.0 ml/min; and detector: ultraviolet (referred to as UV hereinafter) (at 292 nm).

Two liters of the 31-hour culture broth containing 78.7 mg/L of vitamin B₆ was centrifuged at 7,500 rpm for 10 min. The pH of the resultant supernatant was adjusted to 3.1 with 1N hydrochloric acid, and then the supernatant was applied to a column (5.5×15 cm) packed with 350 ml of Amberlite CG 120 (H+ form, 100-200 mesh, Rohm and Haas Company, Philadelphia, Pa., USA). The column was washed with 500 ml of deionized water and then eluted with 5% ammonium hydroxide. The vitamin B₆ fractions were concentrated under reduced pressure. The residue thus obtained was dissolved in 10 ml of deionized water, and the solution was charged on a column (5.5×16 cm) packed with 380 ml of Dowex 1×4 (OH⁻ form, 200400 mesh, Dow Chemical Co., Ltd., Midland, Mich., USA), and then washed with 500 ml of deionized water. The column was then eluted with 0.1 N HCl. The fractions containing pyridoxol were concentrated to small volume under reduced pressure. After the solid residue was dissolved in a small amount of hot ethanol, the solution was kept standing at 4° C. overnight. The resultant precipitates were collected by filtration and dried in vacuum to obtain 128 mg of crude crystals. It was recrystallized from ethanol to obtain 98 mg of white crystals having a melting point of 160° C. The infrared absorption, UV absorption, and NMR spectrum of the product of the product coincided with those of authentic pyridoxol.

1. A recombinant microorganism being capable of producing vitamin B6, wherein said microorganism carries extra genes which code for an enzyme combination selected from:
i) erythrose 4-phosphate dehydrogenase and 1-deoxy-D-xylulose 5-phosphate synthase;
ii) erythrose 4-phosphate dehydrogenase and pyridoxol 5′-phosphate synthase; and
iii) erythrose 4-phosphate dehydrogenase, 1-deoxy-D-xylulose 5-phosphate synthase and pyridoxol 5′-phosphate synthase.

2. The microorganism according to claim 1, wherein said microorganism belongs to the genus Escherichia.

3. A process for preparing vitamin B6 comprising the steps of:
i) culturing the recombinant microorganism of claim 1 in a fermentation broth; and
ii) separating the resulting vitamin B6 from the fermentation broth.

4. A process for preparing vitamin B6 comprising the steps of:
i) culturing a recombinant microorganism carrying an extra gene encoding erythrose 4-phosphate dehydrogenase in expressible form, in a fermentation broth; and
ii) separating the resulting vitamin B6 from the fermentation broth.

5. The process according to claim 4, wherein said microorganism belongs to the genus Escherichia.

6. The process according to claim 3, wherein said microorganism is cultured in a medium containing an assimilable carbon source, a digestible nitrogen source, inorganic salts, and other nutrients necessary for the growth of the microorganism at a pH value in the range of about 5.0 to 9.0, at a temperature in the range of from 10° C. to 40° C., and for 1 day to 7 days under aerobic conditions.

7. The process according to claim 4, wherein said microorganism is cultured in a medium containing an assimilable carbon source, a digestible nitrogen source, inorganic salts, and other nutrients necessary for the growth of the microorganism at a pH value in the range of about 5.0 to 9.0, at a temperature in the range of from 10° C. to 40° C., and for 1 day to 7 days under aerobic conditions.

8. The process according to claim 5, wherein said microorganism is cultured in a medium containing an assimilable carbon source, a digestible nitrogen source, inorganic salts, and other nutrients necessary for the growth of the microorganism at a pH value in the range of about 5.0 to 9.0, at a temperature in the range of from 10° C. to 40° C., and for 1 day to 7 days under aerobic conditions.
